(19) United States
(12) Patent Application Publication
(10) Pub. No.: US 2006/0008454 A1
(43) Pub. Date: Jan. 12, 2006

(54) Use of a compound for enhancing the expression of membrane proteins on the cell surface

(75) Inventors: Michael Freissmuth, Vienna (AT); Tetyana Kirpenko, Vienna (AT); Christian Nanoff, Vienna (AT)

Correspondence Address: FITZPATRICK CELLA HARPER & SCINTO, 30 ROCKEFELLER PLAZA, NEW YORK, NY, 10112, US

(73) Assignee: Dr. Helmut Brunar

(21) Appl. No.: 10/886,202

(22) Filed: Jul. 7, 2004

(51) Int. Cl. A61K 38/46

(52) U.S. Cl. 424/94.6

(57) ABSTRACT

The present invention is directed to the use of a compound stimulating deubiquitinating activity in a cell for the manufacture of a medicament for enhancing the expression of integral membrane proteins on the cell surface. Especially, the invention is directed to the use of such compound for the manufacture of a medicament for the treatment of a disease of condition selected from the group consisting of cystic fibrosis, diabetes insipidus, hypercholesterinaemia and long QT-syndrome-2.

### BACKGROUND OF THE INVENTION

Field of the Invention

Membrane proteins, especially integral membrane proteins, have to be inserted cotranslationally into the endoplasmic reticulum. This occurs via the translocon, which is a channel formed by the Sec61-subunits. During and after synthesis of membrane proteins in the endoplasmic reticulum, they undergo a strict quality control to ensure correct folding before they are transported to their definitive site of action.

Several aspects of this quality control are incompletely understood; nevertheless it is clear that incorrectly folding of a membrane protein is sensed by the machinery of the endoplasmic reticulum (that is by chaperons, presumably). This leads to activation of ubiquitinating enzymes on the cytoplasmic side. These transfer ubiquitin to the cytoplasmic peptide chain of the incorrectly folded protein which is retrotranslocated through the Sec61 channel and degraded by the 26S proteasome (Kostova Z, Wolf D H., 2003, For whom the bell tolls: protein quality control of the endoplasmic reticulum and the ubiquitin-proteasome connection. EMBO J. 22:2309-2317). It has to be stressed that this scheme relies predominantly on observations that were made in Saccharomyces cervisiae. Based on several pieces of experimental evidence, it is, however, reasonable to assume that the higher eukaryotes employ a related machinery to eliminate misfolded proteins.

It has been increasingly appreciated that many human diseases can be linked to mutations, which result in the retention of the aberrant protein in the endoplasmic reticulum (ER). Cystic fibrosis is most commonly cited as the model disease: More than 1000 mutations have been identified in the gene encoding the CFTR (cystic fibrosis transmembrane conductance regulator) (Rowntree R K, Harris A., 2003, The phenotypic consequences of CFTR mutations. Ann Hum Genet. 67:471-485), but the majority of the patients (˜70%) have the ΔF508-mutation of the CFTR.

The resulting protein can function properly, if it reaches the plasma membrane; however, it fails to reach the plasma membrane due to an overprotective ER quality control mechanism (Pasyk E A, Foskett J K., 1995, Mutant (delta F508) cystic fibrosis transmembrane conductance regulator C1-channel is functional when retained in endoplasmic reticulum of mammalian cells. J. Biol. Chem. 270:12347-12350). There are many more examples that lead to defective ER-export of membrane proteins; these include mutations of the V₂-vasopressin receptor (associated with diabetes insipidus; Oksche A, Rosenthal W., 1998, The molecular basis of nephrogenic diabetes insipidus. J. Mol. Med. 76:326-337), of the LDL-receptor (resulting in hypercholesterinaemia; Hobbs H H, Russell D W, Brown M S, Goldstein J L., 1990, The LDL receptor locus in familial hypercholesterolemia: mutational analysis of a membrane protein. Annu Rev Genet. 24:133-170; Jorgensen M M, Jensen O N, Holst H U, Hansen J J, Corydon T J, Bross P, Bolund L, Gregersen N., 2000, Grp78 is involved in retention of mutant low density lipoprotein receptor protein in the endoplasmic reticulum. J. Biol. Chem. 275:33861-33868), or of the HERG-K⁺-channel (resulting in long QT-syndrome-2; Kupershmidt S, Yang T, Chanthaphaychith S. Wang Z, Towbin J A, Roden D M., 2002, Defective human Ether-a-go-go-related gene trafficking linked to an endoplasmic reticulum retention signal in the C terminus. J. Biol. Chem. 277: 27442-27448) etc.

It is unclear why these mutated proteins are retained and eventually degraded although they are—at least in part—functionally active (see Pasyk E A, Foskett J K., 1995, Mutant (delta F508) cystic fibrosis transmembrane conductance regulator C1-channel is functional when retained in endoplasmic reticulum of mammalian cells. J. Biol. Chem. 270:12347-12350). However, the available evidence suggests that the quality control machinery in the endoplasmic reticulum is overprotective.

### SUMMARY OF THE INVENTION

It is an object of the present invention to provide means for enhancing the expression of membrane proteins, especially integral membrane proteins, on the cell surface. Especially, it is an object of the present invention to provide means for enhancing the expression of a protein selected from the group consisting of CFTR (cystic fibrosis transmembrane conductance regulator), V₂-vasopressin receptor, LDL-receptor and HERG-K⁺-channel and, furthermore, to provide a medicament for the treatment of a disease or condition selected from the group consisting of cystic fibrosis, diabetes insipidus, hypercholesterinaemia and long QT-syndrome-2.

This object is achieved by the subject matter of the independent claims. Preferred embodiments are disclosed in the dependent claims.

It has been found that stimulating the deubiquitinating activity in a cell, especially by increasing the amount of deubiquitinating enzymes in the cell or stimulating them, enhances the expression of integral membrane proteins on the cell surface. Apparently, deubiquitinating enzymes are capable of decreasing the level of overprotective quality control in the endoplasmatic reticulum.

Several therapeutic concepts have been proposed that may allow to overcome the stringent quality control (see e.g. Cohen F E, Kelly J W., 2003, Therapeutic approaches to protein-misfolding diseases. Nature 426:905-909). However, enhancing deubiquitinating activity has not yet been proposed as a strategy that would allow for enhanced surface expression of membrane proteins and mutated versions thereof.

Stimulating the deubiquitinating activity in a cell may be accomplished by any means. For example, the cell may be contacted with a compound capable of stimulating the deubiquitinating activity in the cell. Such compounds include, but are not limited to, compounds that increase the expression of deubiquitinating enzymes, compounds that suppress inhibitors of deubiquitinating enzymes, and compounds that stimulate the enzymatic activity of deubiquitinating enzymes.

In a preferred embodiment, increasing the amount of deubiquitinating enzymes in the cell can be achieved especially by introducing into the cell a compound selected from the group consisting of
a deubiquitinating enzyme
a nucleic acid sequence encoding a deubiquitinating enzyme.

Especially, the cell may be transfected with an appropriate plasmid containing DNA encoding the deubiquitinating enzyme, followed by expression of the enzyme in the cell.

The ways to introduce a deubiquitinating enzyme or the nucleic acid sequence coding therefore, as well as identifying suitable amounts of compound to be introduced, are known to the skilled artisan or can be determined using knowledge which is well available to the skilled artisan.

Preferably the deubiquitinating enzyme is selected from the group consisting of ubiquitin carboxy-terminal hydrolases (UCH) and ubiquitin specific proteases (USP). USPs are also being referred to as ubiquitin processing proteases (UBPs; Wing, Simon; 2003, Deubiquitinating enzymes—the importance of driving in reverse along the ubiquitin-proteaseome pathway. IJBCB 35:590-605).

Deubiquitinating enzymes are thiol proteases which hydrolyse the amide bond between Gly76 of ubiquitin and the substrate protein. There are two classes of deubiquitinating enzymes; the ubiquitin-specific processing protease or USP class is one of these two known classes of deubiquitinating enzymes (Papa F. R, Hochstrasser M., 1993, The yeast DOA4 gene encodes a deubiquitinating enzyme related to a product of the human tre-2 oncogene. Nature 366:313-319). While the catalytic activity has been tested using artificial substrates, very little is known about their physiological substrates and thus their physiological functions. USPs have been shown to play a role in determination of cell fate (fat facets; Huang Y, Baker R T, Fischer-Vize J A., 1995, Control of cell fate by a deubiquitinating enzyme encoded by the fat facets gene. Science 270:1828-1831; transcriptional silencing [UBP3; Moazed, D., Johnson, D., 1996, A deubiquitinating enzyme interacts with SIR4 and regulates silencing in S. cerevisiae. Cell 86: 667-677], response to cytokines [DUB1 and 2; Zhu Y, Pless M, Inhorn R, Mathey-Prevot B, D'Andrea A D., 1996, The murine DUB-1 gene is specifically induced by the betac subunit of interleukin-3 receptor. Mol Cell Biol. 16:4808-4817] and oncogenic transformation [tre-2, USP4; Gilchrist, C. A., Baker, R. T., 2000, Characterization of the ubiquitin-specific protease activity of the mouse/human Unp/Unph oncoprotein. Biochim Biophys Acta 1481, 297-309]), but the mechanistic details have remained enigmatic.

In an especially preferred embodiment, the deubiquitinating enzyme is USP-4. The sequence of murine USP-4 enzyme is, for example, disclosed in Strausberg, R. L., et al.; Proc. Natl. Acad. Sci. U.S.A. 99 (26), 16899-16903 (2002). Human USP-4 exists in two variants, cf. Puente, X. S. et al., Nat. Rev. Genet. 4 (7), 544-558 (2003).

Preferably, the medicament for enhancing expression of integral membrane proteins on the cell surface additionally comprises a compound selected from the group consisting of
a proteasome inhibitor and
a nucleic acid sequence encoding a proteasome inhibitor.

It has been found that the additional influence of a proteasome inhibitor in combination with deubiquitinating enzymes amounts to an even more significant expression of the membrane proteins on the cell surface. The fact that proteasome inhibitors may enhance the expression of membrane proteins on the cell surface, is known as such, cf. e.g. Jensen T J et al.; Cell. 1995 Oct. 6; 83(1):129-35.

Preferably, the proteasome inhibitor is MG132. MG132 is a tripeptidaldehyde having the structure leucyl-leucyl-norleucinal (LLnL).

The method of the present invention enables especially expression of a protein selected from the group consisting of CFTR (cystic fibrosis transmembrane conductance regulator), V₂-vasopressin receptor, LDL-receptor and HERG-K⁺-channel.

Furthermore the method of the present invention can be used for the treatment of conditions or diseases related to or associated with the lack of expression of membrane proteins on the cell surface.

Especially, the method of the present invention enables treatment of a disease or condition selected from the group consisting of cystic fibrosis, diabetes insipidus, hypercholesterinaemia and long QT-syndrome-2.

The present invention is also directed to a pharmaceutical composition, comprising a therapeutically effective amount of a compound stimulating deubiquitinating activity in a cell.

Preferably, said compound is selected from the group consisting of
a deubiquitinating enzyme
a nucleic acid sequence encoding a deubiquitinating enzyme.

Furthermore, preferably the pharmaceutical composition according to the present invention additionally comprises a therapeutically effective amount of a compound selected from the group consisting of
a proteasome inhibitor and
a nucleic acid sequence encoding a proteasome inhibitor.

### BRIEF DESCRIPTION OF THE FIGURES

FIG. 1 shows coexpression of A_{2A}-receptor and USP4 in HEK293 cells: HEK293 cells were transiently transfected with the following sets of plasmids: CFP-tagged A_{2A}-receptor (=A_{2A}R) (Figures A, E); CFP-tagged A_{2A}R and GFP-tagged USP4 (Figures B, F); CFP-tagged A_{2A}R(1-311) (Figure C); CFP-tagged A_{2A}R(1-311) GFP-tagged USP4 (Figure D).

FIG. 2 shows deubiquitination of the A_{2A}-receptor by USP4: Immunoprecipitation of the A_{2A}-receptor (A_{2A}R) was carried out from HEK293 cells, transiently transfected with the following sets of plasmids: Flag-tagged A_{2A}R, HA-tagged ubiquitin (lanes 1, 2); Flag-tagged A_{2A}R, HA-tagged ubiquitin and GFP-tagged USP4 (lanes 4,5); GFP-tagged USP4 and/or HA-tagged ubiquitin (lanes 6, 3=control lanes).

FIG. 3A shows saturation isotherms for specific binding of [³H]ZM241385 to membranes from transiently transfected HEK293 cells expressing the full-length A_{2A} receptor:

FIG. 3B shows saturation curves for specific binding of [³H]ZM241385 to membranes from transiently transfected HEK 293 cells expressing the truncated versions of the A_{2A}-receptor [A_{2A}R(1-311) and A_{2A}R(1-360)] with or without USP4.

FIG. 3C shows the summary of Bₘₐₓ values from Panels A & B and saturation experiments done with membranes of cells that had been incubated for 3 h in the absence and presence of the proteasome inhibitor MG132 (50 μM):

FIG. 4 shows the stimulation of cAMP accumulation in transiently transfected HEK293 cells:

FIG. 5 shows saturation curves for specific binding of [³H]ZM241385 to membranes from PC12 cells (that endogenously express the A_{2A}-receptor):

### DETAILED DESCRIPTION OF THE INVENTION

### EXAMPLES

In the following examples the A_{2A}-adenosine receptor was employed as a model protein for the following reasons:

(i) The A_{2A}-adenosine receptor is a prototypical G protein-coupled receptor and thus a representative of a class of >1000 receptors (many of which are of obvious therapeutic interest because they serve as drug targets).

(ii) G protein-coupled receptors have been documented to incur a folding problem; in other words, a large portion of newly synthesized protein (≧50%) is subject to degradation in the endoplasmic reticulum and does not reach the plasma membrane (Petaja-Repo U E, Hogue M, Laperriere A, Walker P, Bouvier M., 2000, Export from the endoplasmic reticulum represents the limiting step in the maturation and cell surface expression of the human delta opioid receptor. J. Biol. Chem. 275:13727-13736; Petaja-Repo U E, Hogue M, Laperriere A, Bhalla S, Walker P, Bouvier M., 2001, Newly synthesized human delta opioid receptors retained in the endoplasmic reticulum are retrotranslocated to the cytosol, deglycosylated, ubiquitinated, and degraded by the proteasome. J. Biol. Chem. 276:4416-4423; Puente, X. S. et al., 2003, Nat. Rev. Genet. 4, 544-558; Pankevych H, Korkhov V, Freissmuth M, Nanoff C., 2003, Truncation of the A₁-adenosine receptor reveals distinct roles of the membrane-proximal carboxyl terminus in receptor folding and G protein coupling. J. Biol. Chem. 278:30283-30293). This is similar to the situation with many other membrane proteins with multiple transmembrane spans, specifically with CFTR (Jensen T J, Loo M A, Pind S, Williams D B, Goldberg A L, Riordan J R., 1995, Multiple proteolytic systems, including the proteasome, contribute to CFTR processing. Cell 83:129-135; Rowntree R K, Harris A., 2003, The phenotypic consequences of CFTR mutations. Ann Hum Genet. 67:471-485).

(iii) There is at least one disease where mutations cause retention of a G protein-coupled receptor in the endoplasmic reticulum: In some instances, diabetes insipidus results from point mutations of the gene encoding the V₂-vasopressin receptor that can be linked to ER-retention of the receptor (Oksche A, Rosenthal W., 1998, The molecular basis of nephrogenic diabetes insipidus. J. Mol. Med. 76:326-337).

Materials and Methods

Radioligand Binding Assays:

Membranes (100 μg/assay) that had been prepared from PC12 cells or HEK293 cells transiently transfected with the appropriate plasmids were incubated in a final volume of 0.3 ml containing 50 mM Tris.HCl (pH 8.0), 1 mM EDTA, 5 mM MgCl2, 8 μg/ml adenosine deaminase and concentrations of [³H]ZM241385 (specific activity ˜20 Ci/mmol) covering the range of 0.2 to 20 nM in the presence of 100 μM GTPγS (Klinger, M., Kuhn, M., Just, H., Stefan, E., Palmer, T., Freissmuth, M., Nanoff, C., 2002, Removal of the carboxy terminus of the A_{2A}-adenosine receptor blunts constitutive activity: Differential effect on cAMP accumulation and MAP kinase stimulation. Naunyn Schmiedeberg's Arch. Pharmacol. 366: 287-298). After 60 min at room temperature, the reaction was terminated by rapid filtration over glass fiber filters. Nonspecific binding was determined in the presence of 10 μM XAC and amounted to 40% at the highest concentration of [³H]ZM241385. The data points were fitted by non-linear regression to the equation describing a rectangular hyperbola. Assays were performed in duplicate.

Agonist Mediated Cellular cAMP Accumulation:

Cells were grown in 6-well plates. The adenine nucleotide pool was metabolically labelled by incubating confluent monolayers for 16 h with [³H]adenine (1 μCi/well) as described (Kudlacek, O., Mitterauer, T., Nanoff, C., Hohenegger, M., Tang, W.-J., Freissmuth, M., and Kleuss, C., 2001, Inhibition of adenylyl and guanylyl cyclase isoforms by the antiviral drug foscarnet. J. Biol. Chem. 276:3010-3016). After the preincubation, fresh medium was added that contained 100 μM RO201724 (a phosphodiesterase inhibitor) and adenosine deaminase (2 U/ml) to remove any endogenously produced adenosine. After 1 h, cAMP formation was stimulated by the A_{2A}-selective agonist CGS21680 (1 nM to 1 μM) for 15 min and the reaction was stopped by adding 2.5% perchloric acid with 100 μM cAMP (1 ml/dish). The supernatant (0.9 ml) was aspirated, neutralized with 100 μl of 0.4 M KOH, and diluted with 1.5 ml 50 mM Tris-HCl, pH 8.0. [³H]cAMP was isolated by sequential chromatography on Dowex AG 50W-X4 and neutral alumina columns (Salomon (1991). Assays were performed in triplicate.

Immunoprecipitation of the Epitope-Tagged A_{2A}-Adenosine Receptor:

HEK293 cells stably expressing FLAG-tagged A_{2A}-adenosine receptor were washed three times with phosphate buffered saline; subsequently, the membranes were solubilized in ice cold lysis buffer [50 mM Tris.HCl, pH 7.5, 1 mM EDTA, 150 mM NaCl containing 1% Nonidet P-40 (vol/vol), protease inhibitors (Complete, Roche Molecular Biochemicals) and, where indicated, 10 mM N-ethylmaleimide (NEM)] for 1 h on ice. The insoluble material was collected by centrifugation at 16,000×g for 10 min at 4° C. The supernatant was processed for immunoprecipitation, each step of which was conducted with constant rotation at 4° C. Then 40 μl of a 50% (vol/vol) suspension of Anti-Flag M2 Affinity Gel (Sigma Chemical) was added and the sample was incubated overnight. The beads were collected by centrifugation and washed three times in 1 mL Tris-buffered saline. Immune complexes were dissociated in SDS-polyacrylamide sample buffer containing 20 mM dithiothreitol by incubation for 1 h at 37° C. or, alternatively, for 5 min at 95° C. Proteins were transferred to nitrocellulose membranes (Immobilon-P, Millipore) by using a semidry transfer system; immunodetection was achieved by using monoclonal peroxidase-conjugated anti-FLAG and anti-HA antibodies to detect the FLAG epitope of the A_{2A}R and the HA-epitope of ubiquitin respectively. The GFP moiety in USP4 was detected with an anti-GFP antiserum (Living colors A.v.) and a horseradish peroxidase conjugated anti-rabbit IgG secondary antibody. The immunoreactive bands were developed with the enhanced chemiluminescence detection kit (Pierce SuperSignal).

Fluorescent Microscopy:

Transiently transfected HEK-293 cells were investigated 1 day after transfection on an inverted epifluorescence microscope (Zeiss Axiovert 200M) using a 63-fold oil immersion objective and filter sets, which discriminate between CFP and YFP fluorescence (Chroma Technology Corp.; Brattleboro, Vt.). Images were captured with a cooled CCD-Kamera (CoolSNAP fx; Photometrics, Roper Scientific, Tucson, Ariz.) and stored in and processed with MetaSeries software (release 4.6 Metafluor and Metamorph; Universal Imaging).

Results

USP4 Enhances the Cell Surface Expression of the A_{2A}-Adenosine Receptor

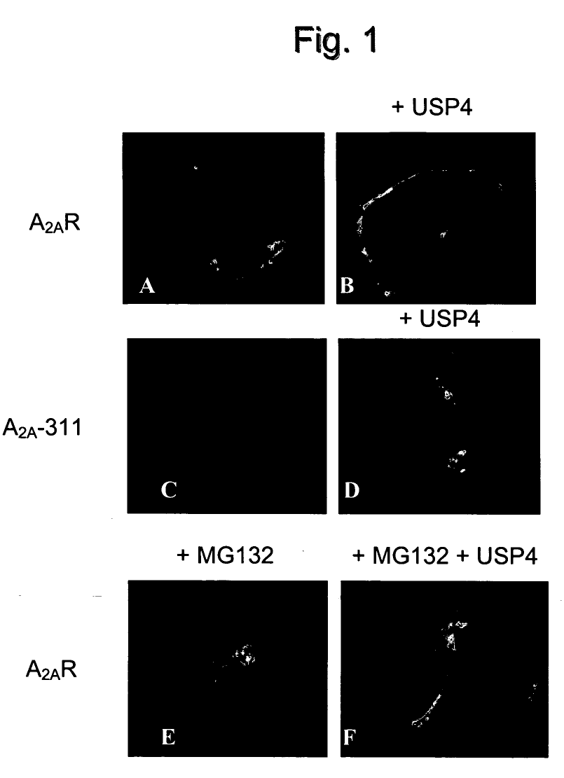

In order to visualize the A_{2A}-adenosine receptor in living cells, the receptor was tagged on its carboxyl terminus with the cyan-fluorescent protein (CFP, a spectrally shifted variant of the green fluorescent protein of Aequoria victoria). This receptor binds ligands and activates its downstream signalling cascade in a manner indistinguishable from the untagged receptor (data not shown). Fluorescent microscopy revealed that, when expressed in HEK293 cells, a large portion of the receptor accumulates within the cell (FIG. 1A).

If the cells are cotransfected with a plasmid driving the expression of the deubiquinating enzyme USP4, the fluorescently tagged A_{2A}-adenosine receptor was found predominantly at the plasma membrane (FIG. 1B).

In the current model, quality control in the endoplasmic reticulum is thought to require ubiquitination of the carboxyl terminus (Kostova and Wolf, 2003). Therefore, it was investigated whether a truncation of the carboxyl terminus of the A_{2A}-receptor ought to render the receptor insensitive to the action of USP4. This was the case: a comparison of FIG. 1C and FIG. 1D shows that the absence and presence of USP4 does not affect the portion of fluorescent receptor that is trapped within the cell.

Finally, it was investigated whether inhibition of proteosomal degradation would, furthermore, relax quality control and thus allow the receptor to escape from the endoplasmic reticulum. The addition of the proteasome inhibitor MG132 did, in fact, augment the amount of receptor at the cell surface (cf. FIG. 1E and FIG. 1A); in the presence of both, USP4 and MG132, essentially all of the receptor was found at the cell surface (FIG. 1F). Cells were incubated in the presence of the proteasome inhibitor MG132 (50 μM) for 3 h (Figures E,F). Images were captured 24 h later with the appropriate filter settings. The experiments were carried out three times with comparable results.

Coexpression of USP4 Results in the Accumulation of Deubiquitinated A_{2A}-Receptor

In order to show that USP4 utilized the A_{2A}-receptor as substrate, HEK293 cells were transiently cotransfected with plasmids encoding for the Flag-tagged A_{2A}-adenosine receptor, HA-tagged ubiquitin and GFP-tagged USP4.

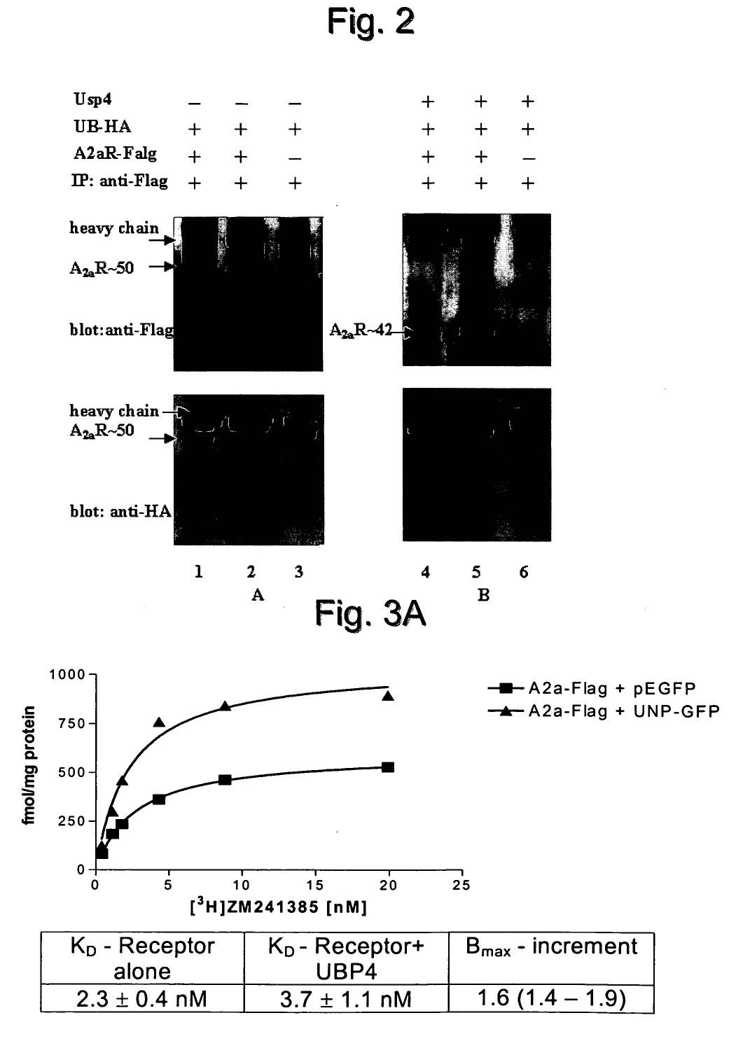

The A_{2A}-adenosine receptor was immunoprecipitated with anti-Flag antibodies from detergent lysates of cells that either coexpressed only HA-tagged ubiquitin (FIG. 2A, lanes 1,2) or the combination of HA-tagged ubiquitin and USP4 (FIG. 2B, lanes 4,5): Receptor bands were detected with anti-Flag antibody (blots shown on top); in the absence of USP4, the FLAG-reactive immunostaining was seen in the range of ˜48-50 kDa (FIG. 2A top, lanes 1,2); in the presence of USP4, the FLAG-tagged receptor migrated at ˜40-42 kDa (FIG. 2B top, lanes 1,2).

Lanes 3 and 6 represent the negative controls, that is immunoprecipitation was carried out with cellular lysates that lacked the A_{2A}-adenosine receptor but contained HA-tagged ubiquitin and—in lane 6—USP4. Regardless of the conditions, immunoreactivity was neither recovered in the ˜40-42 kDa nor in the ˜48-50 kDa range. Thus, the immunostaining was specific.

The nitrocellulose membranes were stripped and stained with anti-HA antibodies (FIGS. 2A&B, bottom blots). In cells cotransfected with the plasmids encoding the Flag tagged A_{2A}-adenosine receptor and HA-tagged ubiquitin, the HA-antibody stained a ˜48-50 kDa band. This corresponded to the ubiquitinated form of A_{2A}-receptor, because this band was also stained with the anti-HA antibody (cf. FIG. 2A top and bottom blots). In contrast, when coexpressed with USP4, the A_{2A}-receptor, which migrated as a band of 40-42 kDa (FIG. 2B, top, lanes 4&5), was not detected with the anti-HA antibody. This band, therefore represents the deubiquitinated species of the receptor. In FIG. 2, cells were collected 48 h after transfection and membrane preparation, immunoprecipitation were done as described above. After the electrophoretic transfer, membranes with proteins were stained with anti-Flag antibody (1:500 dilution) to reveal A_{2A}-receptor immunoreactivity (upper panel), than stripped for 30 min at 50° C. and incubated with anti-HA antibody to stain ubiquitin (lower panel). Data are from a representative experiment that was reproduced 3 times.

Coexpression of USP4 Enhances the Expression of Functional A_{2A}-Receptors

As documented in FIG. 1, USP4 caused a redistribution of the CFP-tagged A_{2A}-receptor to the cell surface. It is conceivable that relaxing quality control by coexpressing USP4 allowed unfolded receptors to escape from the endoplasmatic reticulum.

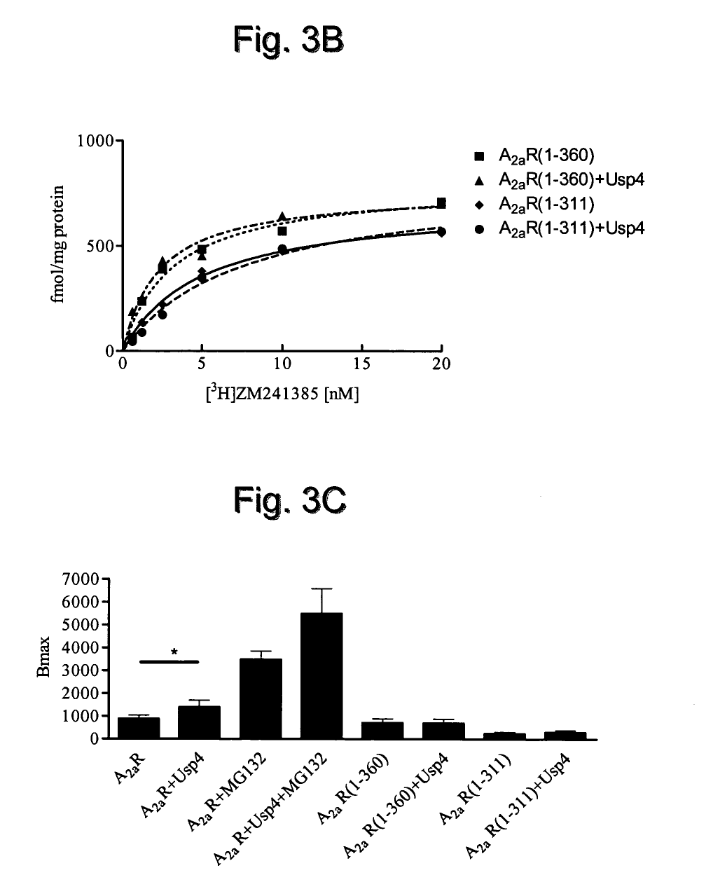

In order to rule out this possibility, binding assays were performed with [³H]ZM241385, a specific and selective A_{2A}-receptor antagonist (Palmer T M, Poucher S M, Jacobson K A, Stiles G L., 1995, ¹²⁵I-4-(2-[7-amino-2-[2-furyl][1,2,4]triazolo[2,3-a][1,3,5]triazin-5-yl-amino]ethyl)phenol, a high affinity antagonist radioligand selective for the A₂ₐ-adenosine receptor. Mol. Pharmacol. 48:970-974.). FIG. 3A shows a set of representative saturation curves for specific binding of [³H]ZM241385 to membranes from HEK293 cells that were either solely transfected with a plasmid driving the expression of (either the CFP or the FLAG-tagged) A_{2A}-receptor or of the receptor and USP4. The coexpression of USP4 (FIG. 3, red symbols) increased Bₘₐₓ but did not affect the affinity of the radioligand. Membranes were prepared from HEK293 cells transfected with plasmids driving the expression of the full-length Flag-tagged A_{2A}-receptor and enhanced green fluorescent protein (pEGFP) or the full-length A_{2A}-receptor and GFP tagged USP4 (=UBP4=ENP-GFP); these membranes were incubated in buffer containing the indicated concentrations of [³H]ZM241385 in the presence of 100 μM GTPgS. Data A&B are means from duplicate determinations in a representative experiment which was repeated three times (the mean parameters are shown in tabulated form). This effect of USP4 depended on the carboxyl terminus of the A_{2A}-receptor, for it was not seen with the truncated forms A_{2A}-receptor-(1-311) or A_{2A}-receptor(1-360), which lack the last 100 and the last 50 amino acids respectively; representative saturation curves are shown in FIG. 3B; Bₘₐₓ averaged from several saturation experiments are shown in the bar diagram in FIG. 3C. Results are means±SD from 4 independent experiments that were carried out in parallel and done with duplicate determinations. Asterisk indicates a significant difference from the full length A_{2A}R at p=0.001 (unpaired t-test).

The model of quality control in the endoplasmatic reticulum leads to the assumption that all steps are reversible provided that the carboxyl terminus of the membrane protein has not yet been engulfed by the proteasome (Kostova and Wolf, 2003). Accordingly, it was investigated whether the action of USP4 and of proteasome inhibition is additive. This was the case. As can be seen from the average Bₘₐₓ-values summarized in FIG. 3C, sole addition of MG132 caused a pronounced increase in the amount of functional receptors, but the combined presence of both, USP4 and MG132 resulted in a dramatic increase in the number of receptors.

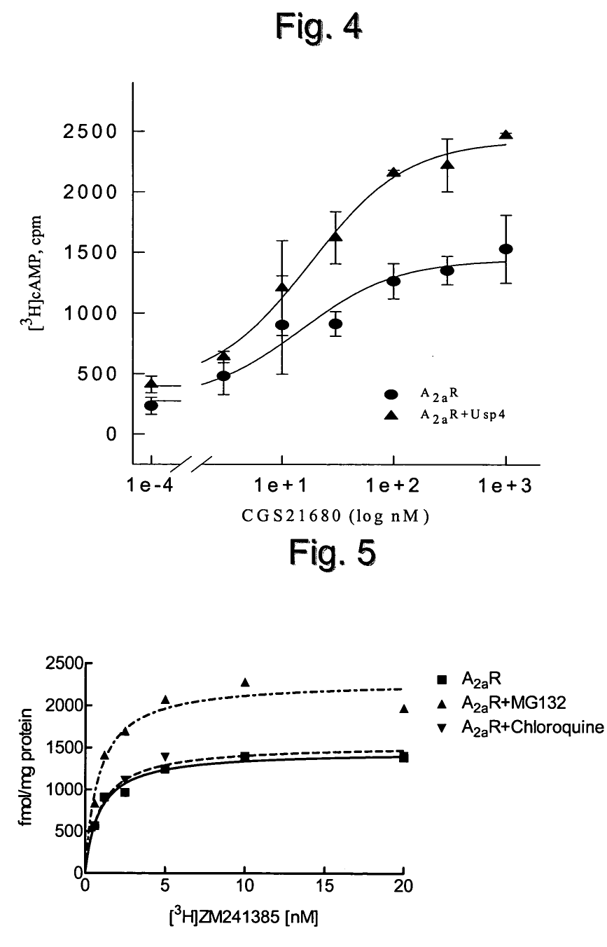

The A_{2A}-adenosine receptor is a prototypical Gs-coupled receptor, thus activation of the receptor leads to stimulation of adenylyl cyclase. The binding data showed that coexpression of USP4 increased the number of functional receptors. This conclusion was verified independently by measuring agonist-induced cellular cAMP accumulation. In cells that expressed USP4, the agonist CGS21680 elicited a larger maximum effect than in cells that only expressed the A_{2A}-adenosine receptor (FIG. 4). It should be noted that this is not a non-specific effect that can, for instance, be accounted for by an increased responsiveness of the catalytic moiety of adenylyl cyclase in the presence of USP4. Control experiments revealed that cells expressing solely the A_{2A}-receptor or the A_{2A}-receptor and USP4 did not differ in their responsiveness to forskolin. In FIG. 4, cells expressing solely the full-length A_{2A}-receptor (circles) or the combination of A_{2A}-receptor and USP4 (triangles) were seeded in 6-well dishes, the cellular adenine nucleotide pool was metabolically prelabelled for 16 h with [³H]adenine. After a preincubation of 30 min in fresh medium containing adenosine deaminase (2 U/ml), cAMP production was stimulated by the indicated concentrations of the A_{2A}-selective agonist CGS 21680. Data are means±SD from 4 independent experiments that were done in triplicate; in each individual experiment, the receptor alone and cotransfected with USP4 were always assayed in parallel.

All experiments shown so far relied on transient transfection to demonstrate the ability of USP4 to enhance the expression of the A_{2A}-receptor. Therefore, also PC12 cells, a rat pheochromocytoma cell line, in which the A_{2A}-receptor is physiologically expressed at high levels, were employed. Addition of the proteasome inhibitor MG132 also resulted in an increase in the membrane concentration of the A_{2A}-receptor (▴ in FIG. 5). In contrast, the lysosomal inhibitor choloroquine did not affect the A_{2A}-receptor levels (▾ in FIG. 5). Membranes were prepared from PC12 cells, which had been incubated in the presence or in the absence of 50 μM MG132 or 100 μM chloroquine for 3 h, and were incubated in buffer containing the indicated concentrations of [³H]ZM241385 in the presence of 100 μM GTPγS.

What is claimed is:

1. A composition comprising a therapeutically effective amount of a compound that stimulates deubiquitinating activity in a cell.

2. The composition according to claim 1, wherein the compound increases the amount of deubiquitinating enzyme in the cell.

3. The composition according to claim 2, wherein said compound is selected from the group consisting of a deubiquitinating enzyme and a nucleic acid sequence encoding a deubiquitinating enzyme.

4. The composition according to claim 3, wherein the deubiquitinating enzyme is selected from the group consisting of ubiquitin carboxy-terminal hydrolases (UCH) and ubiquitin specific proteases (USP).

5. The composition according to claim 4, wherein the USP is USP-4.

6. The composition according to claim 1, 2, 3, 4 or 5, further comprising a therapeutically effective amount of another compound that increases the amount of proteasome inhibitors in the cell.

7. The composition according to claim 6, wherein the another compound is selected from the group consisting of a proteasome inhibitor and a nucleic acid sequence encoding a proteasome inhibitor.

8. The composition according to claim 7, characterized in that the proteasome inhibitor is MG132.

9. The composition according to claim 1, wherein the compound enhances the expression of a protein selected from the group consisting of CFTR (cystic fibrosis transmembrane conductance regulator), V₂-vasopressin receptor, LDL-receptor and HERG-K⁺-channel.

10. A method for enhancing the expression of membrane proteins on a surface of a cell, comprising contacting the cell with a compound that stimulates the deubiquitinating activity in the cell.

11. The method according to claim 10, wherein the compound increases the amount of deubiquitinating enzyme in the cell.

12. The method according to claim 11, wherein the compound is selected from the group consisting of a deubiquitinating enzyme and a nucleic acid sequence encoding a deubiquitinating enzyme.

13. The method according to claim 12, wherein said deubiquitinating enzyme is selected from the group consisting of UCH and USP.

14. The method according to claim 13, wherein the USP is USP-4.

15. The method according to claim 10, 11, 12, 13 or 14, further comprising contacting the cell with another compound that increases the amount of proteasome inhibitors in the cell.

16. The method according to claim 15, wherein the another compound is selected from the group consisting of a proteasome inhibitor and a nucleic acid sequence encoding a proteasome inhibitor.

17. The method according to claim 16, wherein in the proteasome inhibitor is MG132.

18. The method according to claim 10, wherein the compound enhances the expression of a protein selected from the group consisting of CFTR (cystic fibrosis transmembrane conductance regulator), V₂-vasopressin receptor, LDL-receptor and HERG-K⁺-channel.

19. A method for treating a disease or condition selected from the group consisting of cystic fibrosis, diabetes insipidus, hypercholesterinaemia and long QT-syndrome-2, comprising administering to a subject in need thereof an effective amount of a compound that stimulates the deubiquitinating activity in the cell.

20. The method according to claim 10, wherein the compound increases the amount of deubiquitinating enzyme in the cell.

21. The method according to claim 20, wherein the compound is selected from the group consisting of a deubiquitinating enzyme and a nucleic acid sequence encoding a deubiquitinating enzyme.

22. The method of claim 21, wherein the deubiquitinating enzyme is selected from the group consisting of UCH and USP.

23. The method according to claim 22, wherein the USP is USP-4.

24. The method according to claim 19, 20, 21, 22 or 23, further comprising administering to said subject another compound that increases the amount of proteasome inhibitors in the cell.

25. The method according to claim 24, wherein the another compound is selected from the group consisting of a proteasome inhibitor and a nucleic acid sequence encoding a proteasome inhibitor.

26. The method according to claim 25, wherein the proteasome inhibitor is MG132.

27. The method according to claim 19, wherein the compound enhances the expression of a protein selected from the group consisting of CFTR (cystic fibrosis transmembrane conductance regulator), V₂-vasopressin receptor, LDL-receptor and HERG-K⁺-channel.
